(19) United States
(12) Patent Application Publication
(10) Pub. No.: US 2023/0365485 A1
(43) Pub. Date: Nov. 16, 2023

(54) PROCESS FOR PRODUCING LEVULINIC ACID

(71) Applicant: BIO-ON S.P.A., San Giorgio Di Piano (BO) (IT)

(72) Inventors: Simone BEGOTTI, Suzzara (MN) (IT); Alessandro MASSI, Casalecchio di Reno (BO) (IT); Daniele RAGNO, Bologna (IT); Graziano DI CARMINE, Ferrara (IT); Costanza LEONARDI, Cesena (FC) (IT); Olga BORTOLINI, Tavagnacco (UD) (IT)

(21) Appl. No.: 18/028,111

(22) PCT Filed: Sep. 21, 2021
(86) PCT No.: PCT/IB2021/058572

§ 371 Date: Mar. 23, 2023

(30) Foreign Application Priority Data
Sep. 23, 2020 (IT) 102020000022387

(51) Int. Cl. C07C 51/00; C07C 67/00; C07C 51/09

(52) U.S. Cl. CPC C07C 51/00; C07C 67/00; C07C 51/09

(57) ABSTRACT

A process for producing levulinic acid includes a step of catalytic conversion of a pentose (in particular xylose or arabinose) into furfural in an organic solvent having a boiling temperature from 60° C. to 220° C., followed by a step of reduction of furfural to furfuryl alcohol, in the presence of a Lewis acid as catalyst and a protic solvent. Eventually, furfuryl alcohol is converted into levulinic acid directly or indirectly, by preliminary conversion into a levulinic acid ester and its subsequent hydrolysis. This process has a reduced environmental impact and guarantees satisfactory process yields on an industrial scale. In particular, the process allows to reduce as much as possible the formation of humins, which require complex and costly purification processes and involve a considerable reduction in the levulinic acid yields.

### CROSS-REFERENCE TO RELATED APPLICATIONS

This application is a 35 U.S.C. § 371 National Stage patent application of PCT/IB2021/058572 filed 21 Sep. 2021, which claims the benefit of Italian patent application 102020000022387 filed 23 Sep. 2020, the disclosures of which are incorporated herein by reference in their entirety.

### TECHNICAL FIELD

The present disclosure relates to a process for producing levulinic acid. In particular, the present disclosure relates to a process for producing levulinic acid starting from pentoses, i.e. from carbohydrates with 5 carbon atoms.

### BACKGROUND

In recent years, the need has been felt to find new “platform molecules” that can be obtained through production processes that have a lower environmental impact than processes that involve the use of substances of fossil origin. These platform molecules are compounds suitable for the production of other molecules for use in various fields of chemistry.

A molecule useful for this purpose is levulinic acid, which can be used to obtain derivatives that have application in the chemical industry in a broad sense, or, more specifically, in the pharmaceutical, agro-chemical and cosmetic sectors, and in fertilizers, plastics and polymers.

Levulinic acid (also known as 4-oxopentanoic acid or γ-ketovaleric acid) is an organic product with the formula HO—CO—(CH₂)₂—CO—CH₃ that can be widely used in chemical industry, in particular as an intermediate for the production of a wide variety of products such as resins, plasticizers, herbicides, solvents, fuel additives, flavorings, pharmaceuticals, and others.

Currently the most frequently used process for the production of levulinic acid involves the treatment of carbohydrates at acidic pH and at high temperature (up to 200° C.), using a strong acid such as hydrochloric acid or sulfuric acid as a catalyst. For example, starting from sucrose, the most used reaction scheme that leads to formation of levulinic acid is the following:
sucrose (C₁₂H₂₂O₁₁)+H₂O→glucose (C₆H₁₂O₆)+fructose (C₆H₁₂O₆);
fructose (C₆H₁₂O₆)→HMF (C₆H₆O₃)+3 H₂O;
HMF (C₆H₆O₃)+2 H₂O→levulinic acid (C₅H₈O₃)+formic acid (CH₂O₂);
   wherein HMF is 5-(hydroxymethyl)-2-furaldehyde, or 5-(hydroxymethyl)furfural. This mechanism of levulinic acid synthesis is described for example in the article by Meramo-Hurtado, Samir I. et al, ACS Omega 2019, 4, 27, 22302-22312.

However, the yield of this process is actually quite low, mainly due to the formation of numerous reaction by-products from which the levulinic acid must be separated through complex extraction and purification processes. In addition to various low molecular weight by-products, including formic acid, heat treatment of carbohydrates in aqueous solution at acidic pH leads to formation of humins, high molecular weight products resulting from condensation reactions. Humins typically separate as solids, usually dark in color, which cause numerous problems during the recovery process of levulinic acid.

Patent application WO 98/19986 describes a process for producing levulinic acid, which comprises the treatment of a cellulose or hemicellulose-based biomass with a concentrated acid solution. The recovery of levulinic acid is achieved by passing the reaction mixture through a chromatographic column, in particular a system of multiple chromatographic columns, known as “simulated moving bed chromatography”: it is a very slow recovery process that requires the use of complex and expensive equipment.

### SUMMARY

In the state of the art, therefore, a strongly felt need remains to find an alternative process for the production of levulinic acid which has a reduced environmental impact and which guarantees satisfactory process yields on an industrial scale. In particular, one of the main objectives of the present disclosure is to produce levulinic acid by minimizing humins formation, which require complex and costly purification processes and involve a considerable reduction in levulinic acid yields. A further objective of the present disclosure is to carry out this process with the use of eco-compatible reagents and with raw materials from renewable sources.

The Applicant has now found that it is possible to achieve the above objectives and others which will be better illustrated below by means of a process for producing levulinic acid which comprises a catalytic conversion step of a pentose (in particular xylose or arabinose) into furfural in an organic solvent having a boiling temperature from 60° C. to 220° C., followed by a step of reducing furfural to furfuryl alcohol, in the presence of a Lewis acid as catalyst and a protic solvent. Finally, furfuryl alcohol is converted into levulinic acid directly or indirectly, by preliminary conversion into a levulinic acid ester and its subsequent hydrolysis.

The present disclosure therefore relates to a process for producing levulinic acid, which comprises:
(a) converting a pentose into furfural, in the presence of an acid catalyst and an alkali or alkaline earth metal halide, in an organic solvent having a boiling temperature of 60° C. to 220° C., at a temperature of 120° C. at 200° C.;
(b) reducing furfural to furfuryl alcohol, in a protic solvent and in the presence of a Lewis acid as catalyst;
(c) converting furfuryl alcohol into levulinic acid, directly or through conversion of furfuryl alcohol into an alkyl levulinate and subsequent hydrolysis of alkyl levulinate with formation of levulinic acid.

For the purposes of the present disclosure, in the description and claims that follow, the definitions of the numerical ranges include the individual values within the range and its extremes, unless otherwise specified.

### DETAILED DESCRIPTION OF THE DISCLOSURE

For purposes of the present disclosure, in the description and claims that follow, the term “comprising” also includes the terms “which essentially consists of” or “which consists of”.

Step (a) of the process according to the disclosure is carried out in the presence of an acid catalyst and an alkali or alkaline-earth metal halide. The acid catalyst can be of the homogeneous type, i.e. soluble in the reaction environment, or can be of the heterogeneous type, i.e. insoluble in the reaction environment.

In the first case, it is preferable to use an organic acid, in particular methanesulfonic acid CH₃—SO₂—OH (MSA).

Alternatively, an inorganic Brønsted-Lowry acid (for example H₂SO₄, HCl) or a Lewis acid (for example AlCl₃, FeCl₃, ZrCl₄) can be used.

MSA is particularly advantageous, as it guarantees particularly high yields (around 80%). This acid is also advantageous from an environmental point of view, as it is a biodegradable product, halogen-, nitrogen- and phosphorus-free, and having high thermal stability and low corrosiveness. Furthermore, MSA can be easily recovered from the reaction environment by vacuum distillation.

In the case of a heterogeneous acid catalyst, this can consist in particular of an acid zeolite, preferably a βH zeolite.

Step (a) is carried out in an organic solvent having a boiling temperature from 60° C. to 220° C., preferably from 100° C. to 200° C. This solvent allows to operate at relatively high temperatures (from 120° C. to 200° C.) and this allows to significantly reduce the quantity of humins that can form due to secondary reactions involving the starting pentose when it is treated at high temperatures in an acidic environment.

Preferably, the organic solvent is selected from: γ-butyrolactone (GBL), γ-valerolactone (GVL), tetrahydrofuran (THF), dioxane, dimethylsulfoxide (DMS). More preferably, the organic solvent is γ-butyrolactone (GBL). It is a product with eco-compatibility characteristics, that is obtained through non-polluting processes and per se free from harmful effects on the environment.

Preferably, the organic solvent is at least partially miscible with water, so as to allow the reaction to be carried out in the presence of water. The presence of water minimizes the formation of humins and favors conversion to furfural. The organic solvent removes furfural from the aqueous environment, which, in the presence of the catalyst, could decompose, also forming humins. Preferably, the organic solvent is in admixture with water with a weight ratio of organic solvent to water from 50:50 to 98:2, more preferably from 80:20 to 95:5.

As for the halide, this is a halide of an alkali or alkaline earth metal. The halide is preferably chloride, bromide or iodide, more preferably iodide. The alkali metal is preferably selected from: lithium, sodium and potassium, while the alkaline earth metal is preferably selected from: magnesium, calcium, strontium, barium.

Without wishing to bind to an interpretative theory, it is believed that step (a) occurs according to the following mechanism (which is shown starting from xylose as initial pentose):

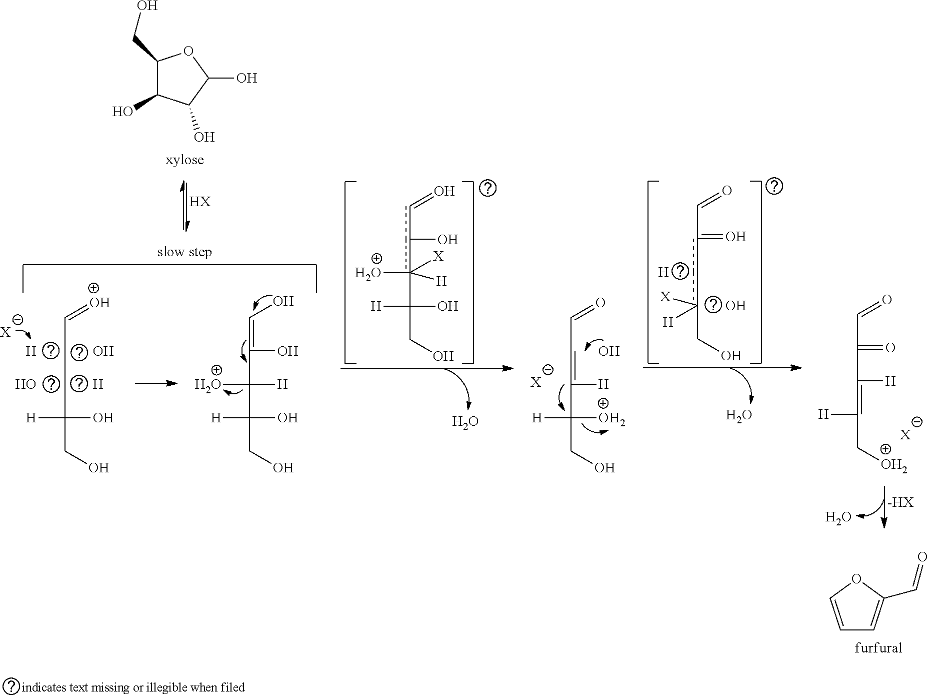

The halide substantially acts as a weak base via proton transfer in the first enolization step, which is the slow sub-step of step (a). During this sub-step the effectiveness of the halide is as follows: Cl>Br>I.

In the following three dehydration sub-steps, it is believed that the halide substantially has the function of stabilizing the two intermediate transition steps that are formed. In this case, the effectiveness of the halide is as follows: I>Br>Cl. Therefore, it may be convenient to use a mixture of two different halides, the first (for example a chloride) more effective in the first sub-step, the second (for example an iodide) more effective in the second sub-step. To avoid the use of a chloride, which poses disposal problems and is therefore not preferable from an environmental point of view, it is particularly convenient to use an iodide, which represents the best compromise of effectiveness for the different sub-steps of step (a).

Preferably, the acid catalyst is used in an amount of from 2% to 40% by weight, more preferably from 5% to 30% by weight, with respect to the weight of pentose.

Preferably, the halide is used in an amount of from 15% to 60% by weight, more preferably from 25% to 50% by weight, with respect to the weight of pentose.

Step (b) of the process is carried out in a protic solvent in the presence of a Lewis acid as catalyst. Preferably, step (b) is carried out in a heterogeneous step, i.e. the Lewis acid is substantially insoluble in the protic solvent.

Lewis acid is preferably selected from: zirconium oxide hydroxide (ZrO(OH)₂), zirconium hydroxide (Zr(OH)₄), zirconium oxide (ZrO₂), aluminum hydroxide (Al(OH)₃), titanium hydroxide (Ti(OH)₄), tin hydroxide (Sn(OH)₄), magnesium hydroxide (Mg(OH)). Preferably, the Lewis acid is in the form of nanoparticles.

Preferably, the Lewis acid is used in an amount of from 5% to 80% by weight, more preferably from 10% to 70% by weight, with respect to the weight of furfural.

Lewis acid can optionally be supported in order to facilitate its recovery, for example on an ion exchange resin, such as an Amberlyst resin (acid sulphonic resin).

The protic solvent is preferably a C₁-C₆ alcohol, more preferably a C₃-C₆ secondary alcohol. Particularly preferred are isopropanol and isobutanol. Secondary alcohols act not only as solvents but also as reducing species (hydride donors), oxidized to the corresponding ketone, which is easily removed from the reaction environment.

Step (b) is preferably carried out at a temperature from 30° C. to 150° C., more preferably from 50° C. to 100° C.

Step (b) is believed to occur according to the Meerwein-Ponndorf-Verley (MPV) mechanism (see, for example, Boronat M. et al, J. Phys. Chem. B 2006, 110, 42, 21168-21174-doi.org/10.1021/jp063249x).

This step (b) is particularly advantageous as it allows reduction of furfural to furfuryl alcohol to be achieved without using the common reducing agents based on hydrides and molecular hydrogen, ensuring a safe and sustainable process from an environmental point of view.

As for step (c), furfuryl alcohol can be directly converted into levulinic acid. Preferably, this conversion is carried out in the presence of an acid catalyst in an aqueous medium, in a homogeneous or heterogeneous phase.

In the case of homogeneous acid catalysis, it is preferable to use a Brønsted-Lowry acid in an aqueous medium, preferably selected from phosphoric acid (H₃PO₄) and methanesulfonic acid (MSA). MSA is particularly preferred, as it guarantees particularly high yields (around 80%).

Step (c) is preferably carried out at a temperature from 80° C. to 160° C., preferably from 100° C. to 150° C.

Preferably, the acid catalyst is used in an amount of from 30% to 120% by weight, more preferably from 70% to 100% by weight, with respect to the weight of furfuryl alcohol.

In the case of heterogeneous catalysis, a Lewis acid, for example AlCl₃, FeCl₃, ZrCl₄, or a Brønsted-Lowry acid, for example sulphonic silica (SiO₂—SO₃H), ion exchange resins, such as an Amberlyst resin (acid sulphonic resin), acid zeolites (e.g. βNH₄⁺ zeolite, βH zeolite, ZSM-5 zeolite), sulphonated activated carbons (AC—SO₃H).

As an alternative to direct conversion of furfuryl alcohol to levulinic acid in an aqueous medium, it is possible to obtain levulinic acid from furfuryl alcohol via a two-step process:
(c1) esterifying furfuryl alcohol with a C₁-C₄ alcohol to obtain an alkyl levulinate (levulinic acid alkyl ester);
(c2) hydrolyzing alkyl levulinate with the formation of levulinic acid.

Step (c1) is preferably carried out in the absence of water, so as to favor the formation of the ester and avoid secondary reactions that can lead to the formation of humins. Preferably, the C1-C4 alcohol is selected from: methanol, ethanol, propanol, butanol. Step (c2) is instead carried out in an aqueous environment, being a hydrolysis reaction.

Both steps (c1) and (c2) are preferably carried out in the presence of an acid catalyst (in homogeneous or heterogeneous phase), at a temperature from 80° C. to 160° C., more preferably from 100° C. to 150° C. The acid catalyst (the same or different for the two steps) can be selected from those indicated above for the direct conversion reaction of furfuryl alcohol to levulinic acid.

As regards the pentose used as initial substrate for the process of the present disclosure, this can be preferably selected from arabinose and xylose. These are products widely available in nature, constituting the basic monomer units of hemicellulose, a constituent of wood together with cellulose and lignin. Indicatively, in dry wood, cellulose represents 30-45%, lignin 20-30% and hemicellulose 10-25%.

It is also possible to obtain the pentose starting from a hexose or a mixture of hexoses. For example, the arabinose can be obtained from glucose, fructose and/or sucrose by removing a carbonaceous unit in the presence of PH zeolites, producing arabinose and formaldehyde as a by-product (see for example Jinglei Cui et al, Green Chem., 2016, 18, 1619-1624 and Luxin Zhang et al, Chem. Eng. J., 2017, 307, 868-876). The βH zeolite itself is able to dehydrate arabinose in situ to produce furfural, which leads to obtain levulinic acid in accordance with the process of the present disclosure.

Therefore, the process according to the present disclosure preferably comprises a preliminary step (a0), preceding step (a), in which a hexose selected from sucrose, fructose and glucose, or mixtures thereof, is subjected to removal of a carbonaceous unit in the presence of a zeolite (in particular a βH zeolite or a βFe zeolite) to obtain arabinose, which is converted in situ to furfural. The latter is then subjected to the subsequent steps (b) and (c).

Preferably, step (a0) is carried out in an organic solvent selected from those indicated above for step (a), optionally mixed with water. Particularly preferred is γ-butyrolactone (GBL). The reaction temperature is preferably between 140° C. and 200° C., more preferably between 160° C. and 190° C.

Step (aG) allows avoiding the formation of HMF (2,5-(hydroxymethyl)furfural) which is normally formed from hexoses and produces large quantities of humins, which, as already pointed out, constitute a significant problem from the operational point of view with considerable reductions in final yield.

The process according to the present disclosure can be summarized by the following general scheme, specifically referring to the two preferred pentoses, namely arabinose and xylose:

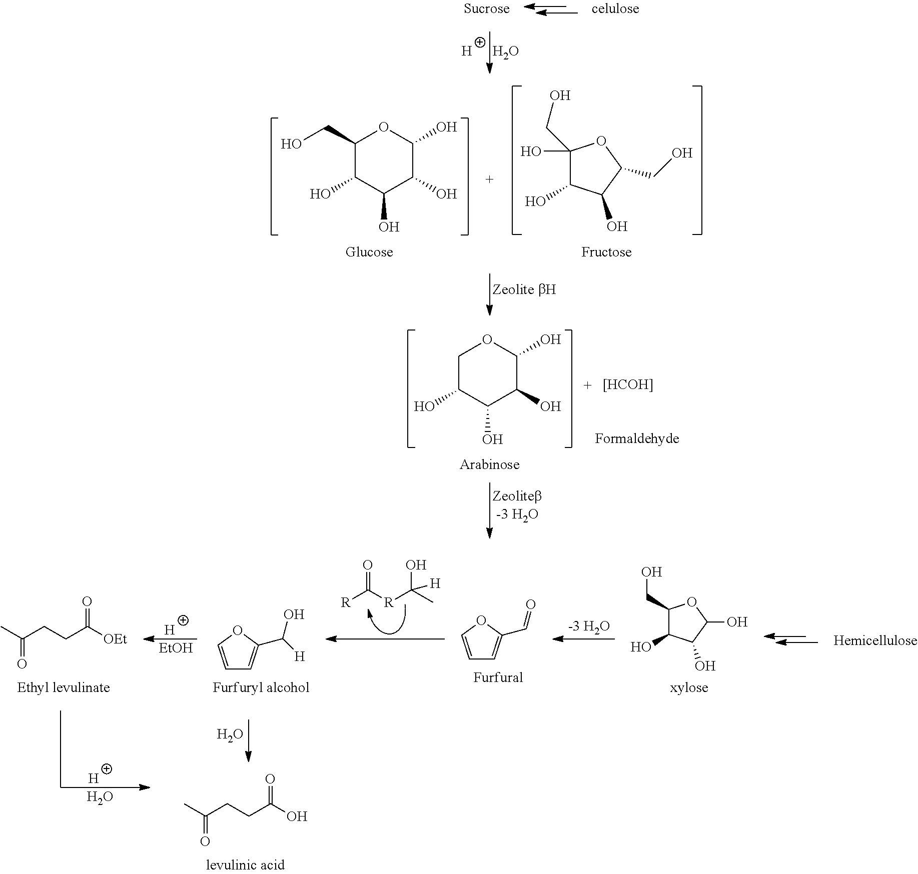

The following examples are intended for illustrative and not limitative purposes of the present disclosure.

### Example 1: Conversion of Xylose to Furfural

(a) Homogeneous Catalysis.

A steel batch reactor equipped with magnetic stirrer was loaded with xylose (1.00 g), γ-butyrolactone (GBL, 16.8 mL), distilled water (1.0 mL; GBL/H₂O 95/5 w/w), potassium iodide (250 mg, 25% w/w) and methanesulfonic acid (200 mg, 20% w/w). Subsequently the reactor was sealed and the reaction mixture heated under stirring (200 rpm) at 150° C. for 3 h. Once the reaction was complete, the reactor was cooled to room temperature and the reaction mixture filtered (porosity 0.45 μm) to obtain a clear solution. Molar yield (80%) and conversion (95%) were determined by analyzing a sample of the reaction mixture by HPLC method.

(b) Heterogeneous Catalysis.

A steel batch reactor equipped with magnetic stirrer was loaded with xylose (1.00 g), γ-butyrolactone (16.8 mL), distilled water (1.0 mL; GBL/H₂O 95/5 w/w), potassium iodide (500 mg, 50% w/w) and zeolite-3H (100 mg, 10% w/w). Subsequently the reactor was sealed and the reaction mixture heated under stirring (200 rpm) at 180° C. for 2 h. Once the reaction was complete, the reactor was cooled to room temperature and the reaction mixture centrifuged (5000 rpm) and filtered (porosity 0.45 μm) to obtain a clear solution. Molar yield (52%) and conversion (98%) were determined by analyzing a sample of the reaction mixture by HPLC method.

The scheme of the two reactions is as follows:

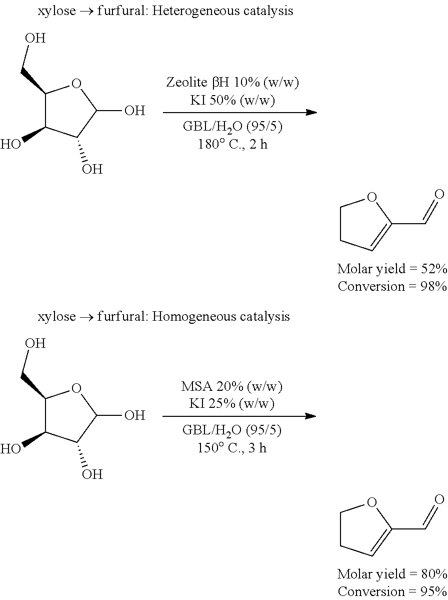 [SMILES: OC[C@H]1OC(O)[C@H](O)[C@H]1O ; O=CC1=CCCO1 ; OC[C@H]1OC(O)[C@H](O)[C@H]1O ; O=CC1=CCCO1]

### Example 2: Reduction of Furfural to Furfuryl Alcohol (Heterogeneous Catalysis)

A steel batch reactor equipped with a magnetic stirrer was loaded with furfural (1.00 g), isopropanol (20 mL) and zirconium oxide hydroxide (500 mg, 50% w/w). Subsequently the reactor was sealed and the reaction mixture heated under stirring (200 rpm) at 80° C. for 24 h. Once the reaction was complete, the reactor was cooled to room temperature and the reaction mixture centrifuged (5000 rpm) and filtered (porosity 0.45 μm) to obtain a clear solution. Molar yield (100%) and conversion (100%) were determined by analyzing a sample of the reaction mixture by HPLC method.

The scheme of the reaction is as follows:

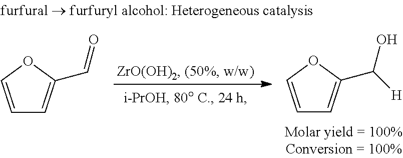 [SMILES: O=Cc1ccco1 ; OCc1ccco1]

### Example 3: Direct Conversion of Furfuryl Alcohol to Levulinic Acid

(a) Homogeneous Catalysis.

A steel batch reactor equipped with magnetic stirrer was charged with furfuryl alcohol (1.00 g), THF (20 mL), distilled water (5.0 mL; THF/H₂O 4/1 v/v), and methanesulfonic acid (1.00 g, 100% w/w). Subsequently the reactor was sealed and the reaction mixture heated under stirring (200 rpm) at 140° C. for 12 h. Once the reaction was complete, the reactor was cooled to room temperature and the reaction mixture filtered (porosity 0.45 μm) to obtain a clear solution. Molar yield (82%) and conversion (100%) were determined by analyzing a sample of the reaction mixture by HPLC method.

(b) Heterogeneous Catalysis.

A steel batch reactor equipped with magnetic stirrer was charged with furfuryl alcohol (1.00 g; 0.125 M), distilled water (81.6 mL) and Amberlyst 15 (24.5 g, 12 equiv. of a resin with 5 mmol/g loading). Subsequently the reactor was sealed and the reaction mixture heated under stirring (200 rpm) at 120° C. for 1.5 h. Once the reaction was complete, the reactor was cooled to room temperature and the reaction mixture centrifuged (5000 rpm) and filtered (porosity 0.45 μm) to obtain a clear solution. Molar yield (48%) and conversion (100%) were determined by analyzing a sample of the reaction mixture by HPLC method.

The scheme of the two reactions is as follows:

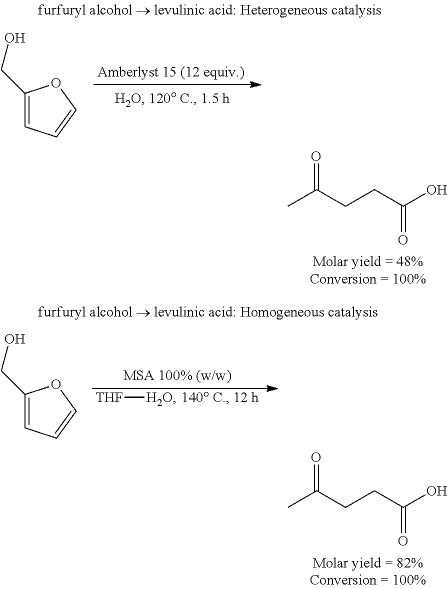

### Example 4: Conversion of Furfuryl Alcohol to Ethyl Levulinate

(a) Homogeneous Catalysis.

A steel batch reactor equipped with a magnetic stirrer was charged with furfuryl alcohol (1.00 g), ethanol (30 mL) and methanesulfonic acid (1.00 g, 100% w/w). Subsequently the reactor was sealed and the reaction mixture heated under stirring (200 rpm) at 120° C. for 12 h. Once the reaction was complete, the reactor was cooled to room temperature and the reaction mixture filtered (porosity 0.45 μm) to obtain a clear solution. Molar yield (95%) and conversion (100%) were determined by analyzing a sample of the reaction mixture by quantitative GC method.

(b) Heterogeneous Catalysis.

A steel batch reactor equipped with a magnetic stirrer was charged with furfuryl alcohol (1.00 g), ethanol (30 mL) and βNH₄⁺ zeolite (2.00 g, 200% w/w). Subsequently the reactor was sealed and the reaction mixture heated under stirring (200 rpm) at 120° C. for 8 h. Once the reaction was complete, the reactor was cooled to room temperature and the reaction mixture centrifuged (5000 rpm) and filtered (porosity 0.45 μm) to obtain a clear solution. Molar yield (78%) and conversion (100%) were determined by analyzing a sample of the reaction mixture by quantitative GC method.

The scheme of the two reactions is as follows:

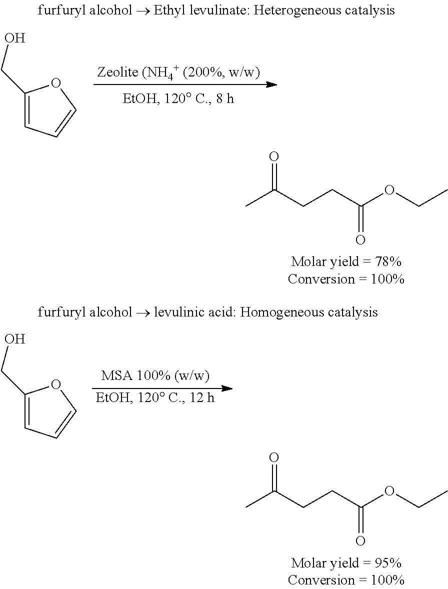

### Example 5: Hydrolysis of Ethyl Levulinate to Obtain Levulinic Acid

(a) Homogeneous Catalysis

A steel batch reactor equipped with a magnetic stirrer was charged with ethyl levulinate (1.00 g), distilled water (30 mL) and methanesulfonic acid (153 mg, 15.3% w/w). Subsequently the reactor was sealed and the reaction mixture heated under stirring (200 rpm) at 120° C. for 18 h. Once the reaction was complete, the reactor was cooled to room temperature and the reaction mixture filtered (porosity 0.45 μm) to obtain a clear solution. Molar yield (95%) and conversion (95%) were determined by analyzing a sample of the reaction mixture by HPLC method.

(b) Heterogeneous Catalysis.

A steel batch reactor equipped with a magnetic stirrer was loaded with ethyl levulinate (1.00 g), distilled water (30 mL) and βH zeolite (153 mg, 15.3% w/w). Subsequently the reactor was sealed and the reaction mixture heated under stirring (200 rpm) at 120° C. for 18 h. Once the reaction was complete, the reactor was cooled to room temperature and the reaction mixture centrifuged (5000 rpm) and filtered (porosity 0.45 μm) to obtain a clear solution. Molar yield (90%) and conversion (95%) were determined by analyzing a sample of the reaction mixture by HPLC method.

The scheme of the two reactions is as follows:

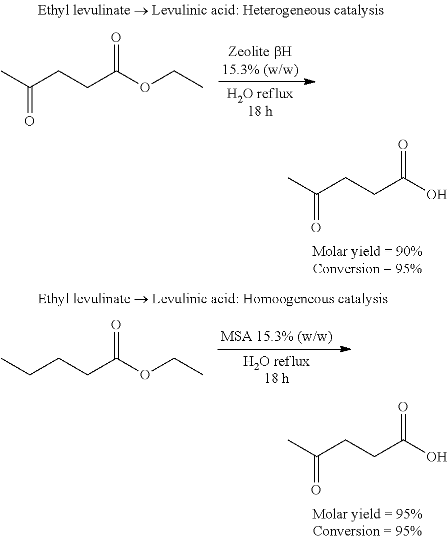

### Example 6: Conversion of Sucrose into Furfural Through Arabinose Formation (Heterogeneous Catalysis)

A steel batch reactor equipped with magnetic stirrer was loaded with sucrose (1.00 g), γ-butyrolactone (16.8 mL), distilled water (1.0 mL; GBL/H₂O 95/5 w/w), potassium iodide (500 mg, 50% w/w) and pH-zeolite (100 mg, 10% w/w). Subsequently the reactor was sealed and the reaction mixture heated under stirring (200 rpm) at 180° C. for 2 h. Once the reaction was complete, the reactor was cooled to room temperature and the reaction mixture centrifuged (5000 rpm) and filtered (porosity 0.45 μm) to obtain a clear solution. Molar yield (65%) and conversion (88%) were determined by analyzing a sample of the reaction mixture by HPLC method.

The scheme of the reaction is as follows:

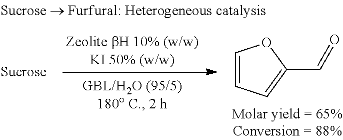

1. A process for producing levulinic acid, the process including the following steps:
(a) converting a pentose into furfural, in the presence of an acid catalyst and of an alkali or alkaline earth metal halide, in an organic solvent having a boiling temperature from 60° C. to 220° C., at a temperature from 120° C. to 200° C.,
(b) reducing furfural to furfuryl alcohol, in a protic solvent and in the presence of a Lewis acid as catalyst, and
(c) converting furfuryl alcohol into levulinic acid, either directly or by converting furfuryl alcohol into an alkyl levulinate and subsequent hydrolysis of alkyl levulinate with formation of levulinic acid.

2. The process according to claim 1, wherein the acid catalyst of step (a) is an organic acid.

3. The process according to claim 1, wherein the acid catalyst of step (a) is a Brønsted-Lowry inorganic acid or a Lewis acid.

4. The process according to claim 1, wherein the acid catalyst of step (a) is an acid zeolite.

5. The process according to claim 1, wherein in step (a) the organic solvent is selected from the group consisting of: γ-butyrolactone (GBL), γ-valerolactone (GVL), tetrahydrofuran (THF), dioxane, and dimethylsulfoxide (DMS).

6. The process according to claim 1, wherein in step (a) the organic solvent is in admixture with water.

7. The process according to claim 1, wherein in step (a) the halide is an iodide.

8. The process according to claim 1, wherein step (b) is carried out in heterogeneous phase, i.e. the Lewis acid is substantially insoluble in the protic solvent.

9. The process according to claim 8, wherein in step (b) the Lewis acid is selected from the group consisting of: zirconium oxide hydroxide (ZrO(OH)₂), zirconium hydroxide (Zr(OH)₄), zirconium oxide (ZrO₂), aluminum hydroxide (Al(OH)₃), titanium hydroxide (Ti(OH)₄), tin hydroxide (Sn(OH)₄), and magnesium hydroxide (Mg(OH)₂).

10. The process according to claim 1, wherein the protic solvent of step (b) is a C₁-C₆ alcohol.

11. The process according to claim 1, wherein step (b) is carried out at a temperature from 30° C. to 150° C.

12. The process according to claim 1, wherein in step (c) the furfuryl alcohol is directly converted into levulinic acid in the presence of an acid catalyst in an aqueous medium, in a homogeneous or heterogeneous phase.

13. The process according to claim 12, wherein the acid catalyst is a Brønsted-Lowry acid.

14. The process according to claim 12, wherein the acid catalyst is a Lewis acid, for example AlCl₃, FeCl₃, ZrCl₄, or a Brønsted-Lowry acid, for example sulphonic silica (SiO₂—SO₃H), ion exchange resins, acid zeolites, sulphonated activated carbons (AC—SO₃H).

15. The process according to claim 1, wherein step (c) is carried out at a temperature from 80° C. to 160° C.

16. The process according to claim 1, wherein in step (c) furfuryl alcohol is converted to levulinic acid in two steps:
(c1) esterifying furfuryl alcohol with a C₁-C₄ alcohol to obtain an alkyl levulinate (levulinic acid alkyl ester), and
(c2) hydrolyzing the alkyl levulinate with formation of levulinic acid.

17. The process according to claim 16, wherein step (c1) is carried out in the absence of water.

18. The process according to claim 16, wherein step (c2) is carried out in an aqueous environment.

19. The process according to claim 16, wherein both steps (c1) and (c2) are carried out in the presence of an acid catalyst, at a temperature from 80° C. to 160° C.

20. The process according to claim 1, wherein in step (a) the pentose is selected from arabinose and xylose.

21. The process according to claim 1, which comprises a preliminary step (a0), preceding step (a), wherein a hexose selected from the group consisting of: sucrose, fructose, and glucose, or mixtures thereof, is subjected to removal of a carbonaceous unit in the presence of a zeolite to obtain arabinose, which is converted in situ to furfural.
